(19) United States
(12) Patent Application Publication
(10) Pub. No.: US 2017/0000039 A1
(43) Pub. Date: Jan. 5, 2017

(54) ENERGY GRASS COMPREHENSIVE RECYCLING METHOD

(71) Applicant: FUJIAN AGRICULTURE AND FORESTRY UNIVERSITY, Fuzhou, Fujian (CN)

(72) Inventors: Bin LIU, Fuzhou, Fujian (CN); Zheng XIAO, Fuzhou, Fujian (CN); Chao ZHAO, Fuzhou, Fujian (CN); Yifan HUANG, Fuzhou, Fujian (CN)

(73) Assignee: FUJIAN AGRICULTURE AND FORESTRY UNIVERSITY, Fuzhou, Fujian (CN)

(21) Appl. No.: 15/039,846

(22) PCT Filed: Jan. 22, 2015
(86) PCT No.: PCT/CN2015/071285

§ 371 Date: May 26, 2016

(30) Foreign Application Priority Data
Jan. 28, 2014 (CN) 201410041568.8

(51) Int. Cl. A01G 1/04; A23K 50/75; A23K 50/10; C12P 5/02; A23K 50/50; A23K 10/12; C05F 3/00; C05F 17/00; A23K 10/30; A23K 50/30

(52) U.S. Cl. CPC A01G 1/04; A23K 10/30; A23K 50/75; A23K 50/10; A23K 50/30; A23K 50/50; A23K 10/12; C05F 3/00; C05F 17/0036; C12P 5/023

(57) ABSTRACT

This present invention discloses an integrated method of cyclic utilization of energy grasses comprising the steps of generating biogas residue from the energy grasses, growing edible mushrooms from the biogas residue, producing livestock or poultry feeds from the spent mushroom culture medium, feeding livestock with the feeds, and producing organic fertilizers from the manure of the livestock or poultry. The method, by producing biogas, edible mushrooms, feeds, livestock or poultry, and organic fertilizers, makes an integrated and cyclic use of energy grasses and aforementioned products. This invention will help achieve the goal of cyclic use of energy grasses with great efficiency.

### FIELD AND BACKGROUND OF THE INVENTION

This present invention, wherein an integrated method of cyclic utilization of energy grasses is disclosed, belongs to the field of agriculture production system.

Energy grass is a class of annual or perennial gramineous plants with high-yielding and high cellulose content. It is widely used in the field of biomass energy. However, its application has not yet fully explored and its utilization is very limited. As such, effort to recognize and increase the economic value of energy grass is a worthy undertaking.

### SUMMARY OF THE INVENTION

The present invention discloses an integrated method of cyclic utilization of energy grasses to integrate the elements of “energy grass-biogas-edible mushrooms-feeds-fertilizer” into a high-efficiency agricultural resource recycling system that produces biogas, edible mushrooms, livestock or poultry feeds, livestock or poultry, and organic fertilizer.

The disclosed method comprises the following steps featuring the cyclic and integrated use of energy grasses, biogas, edible mushrooms, livestock or poultry feeds, and organic fertilizers:

(1) generating biogas and biogas residue from said energy grasses;

(2) growing edible mushrooms from said biogas residue;

(3) producing livestock or poultry feeds from said spent mushroom culture medium;

(4) feeding livestock or poultry with said livestock or poultry feeds;

(5) producing organic fertilizers from the faeces of said livestock or poultry.

The energy grass is selected from one or more of the following:

Neyraudia reynaudiana (Kunth) Keng, Saccharum arundinaceum, Phragmites australis (Cav.) Trin. ex Steud, Miscanthus floridulus (Labill.) Warb. ex Schum. et Laut., Pennisetum purpureum Schumach, Pennisetum sinese Roxb, Pennisetum purpureum cv. Jujuncao, Pennisetum alopecuroides (L.) Spreng, Spartina anglica Hubb., Sorghumpropinqaum (Kunth.) Hitch., Sorghum bicolor L. Moench, silaged Zea mays L, Cymbopogon citratus, Spartina alterniflora Loisel.

The edible mushroom is one of the following: Pleurotus eryngii, Flammulina velutipes, Volvariella volvacea, Pleurotus ostreatus, Agaricus bisporus, Auricularia auricula, Lentinus edodes, Pleurotus geesteranus, Auricularia polytricha, Tremella fucitormis, Ganoderma lucidum, Hypsizygus marmoreus, Grifola frondosa, and Agrocybe cylindracea.

The livestock is one of the following: cattle, sheep, chicken, duck, goose, rabbits, and pigs.

The above steps may be accomplished in the following more specific manners:

(1) generating biogas and biogas residue accomplished by first cutting, smashing and ensiling said energy grasses, and then fermenting the energy grasses to generate biogas and biogas residue;

(2) growing edible mushrooms accomplished by first drying said biogas residue, preparing growth medium from the dry biogas residue, and then growing edible mushrooms using said growth medium;

(3) producing livestock or poultry feeds by fermenting, drying, smashing, and sifting the mushroom residue;

(4) feeding livestock or poultry with said mushroom residue livestock or poultry feeds alone or mixed with other feeds;

(5) producing organic fertilizers accomplished by first mixing said livestock or poultry manure with other organic matters, composting, retting, and inoculating leavening agent into the mixture, and finally fermenting, drying, smashing, sifting, and spraying nutrient fortified composition to obtain organic fertilizers.

More specifically, the method is carried out in the following procedure:

Step 1: producing biogas by energy grass, which comprises the following sub-steps:

1a. cutting energy grasses into 1-6 cm in size, adding silage inoculants, and ensiling energy grasses for more than 15 days;

1b. producing biogas by fermenting said ensiled energy grasses from step 1a under the temperature of 30-60° C., gradually adding ensiled energy grasses to a total dry content of 5-25 wt. %, feeding ensiled energy grasses and discharging biogas residue by 5-40 wt. % matter once every 1-10 days;

Step 2: producing edible mushrooms from the biogas residue from step 1, which comprises the following sub-steps:

2a. taking the biogas residue after 15-45 day normal fermentation from step 1, then extruding or drying the residue to a matter with a water content of 20-60 wt. %;

2b. preparing edible mushrooms culture medium from the biogas residue from step 2a by mixing the culture medium materials, adjusting the water content to 50-70 wt. %, and proceeding to sterilization and inoculation;

2c. producing edible mushrooms with culture medium from step 2b;

Step 3: producing livestock or poultry feed from the spent mushroom culture medium from step 2, which comprises the following sub-steps:

3a. after harvesting mushrooms 1-3 times, inoculating the spent mushroom culture medium with feed microbial agents, fermenting the granules for 5-15 days, and drying the fermented matter to 10-30 wt. % moisture content, then pulverizing the matter and sifting it through a sieve with mesh size of 30-120;

3b. producing livestock feeds from the treated mushroom culture medium from step 3a;

Step 4: feeding livestock or poultry exclusively with the feed from step 3, or supplementing other feeds with 10-40 wt. % of the feed from step 3;

Step 5: producing organic fertilizer from the manure of the livestock or poultry of step 4, which comprises the following sub-steps:

5a. mixing 50%-70% of the manure of the livestock or poultry of step 4 with 10%-45% dry weight of other organic matters, composting and retting the mixture, maintaining the water content to 50-75wt. %;

5b. inoculating organic fertilizer starter culture into the mixture from 5a;

5c. fermenting the mixture for 10-45 days, drying the matter to a moisture content of 5-30 wt. %, pulverizing and sifting the matter through a sieve with mesh size of 30-50, spraying nutrient fortified composition, granulating by disk granulation or extrusion granulation method, and packaging.

Microbes contained in silage inoculants should at least have three functions: degradation of cellulose, degradation of hemicellulose, and production of lactic acid.

The culture medium formula for edible mushrooms which be calculated based on dry base: biogas residue 30%-85%, other organic matter 0-65%, gypsum or lime 0.5%-2.5%, sucrose 0-2%, nitrogen 0-5%, phosphate 0-3%. Said other organic matter comprises one or more of the following: cotton seed hull, rapeseed hull, sawdust, livestock or poultry manure, crop stalks, and corn cobs.

The said other organic matters in step 5 comprise one or more of the following: cotton seed hull, rapeseed hull, spent mushroom culture medium, energy grasses, and corn cobs.

The present invention offers the following significant advantages:

1) We make full use of energy grasses, combine “energy grass-biogas-edible mushrooms-feeds-fertilizer” into a high-efficiency integrated agricultural recycling system, which can produce biogas, edible mushrooms, feeds, livestock or poultry, and fertilizer, and achieve a goal of highly efficient resource recycling.

2) This invention employs an environment-friendly process, in which process steps are so designed that a subsequent step takes the byproducts of a prior step without generating undesirable wastes.

### DESCRIPTION OF THE PREFERRED EMBODIMENTS

The present invention is further described in the following exemplified embodiment to illustrate the application of the principles of the invention. It is understood that the invention may be embodied otherwise without departing from such principles. The scope of the claims of the present invention expressly should not be limited to such exemplary or preferred embodiments.

### Embodiment One

This embodiment discloses a method to combine the elements of “Pennisetum purpureum cv. Jujuncao-biogas-Pleurotus eryngii-feed-cattle-organic fertilizer” into an integrated agricultural recycling system.

The method is carried out in the following procedure:

Step 1: producing biogas from Pennisetum purpureum cv. Jujuncao, which comprises the following sub-steps:

1a. cutting Pennisetum purpureum cv. Jujuncao into 3 cm in size, adding silage inoculants, and ensiling for 20 days;

1b. producing biogas by fermenting said silage from step la under the temperature of 37° C., gradually adding silage to total dry silage content of 15 wt. %, feeding and discharging 20 wt. % matters once every 5 days;

Step 2: producing Pleurotus eryngii from the biogas residue from step 1, which comprises the following sub-steps:

2a. taking the biogas residue after 30 day normal fermentation from step 1, then extruding or drying the residue to a matter with a water content of 60 wt. %;

2b. preparing edible mushrooms culture medium from the biogas residue from step 2a, the culture medium formula containing, on dry base, biogas residue 60%, sawdust 25%, gypsum 2%, sucrose 1.5%, ammonium bicarbonate 2%, and Calcium superphosphate 1%;

2c. mixing the culture medium materials, adjusting the water content to 65 wt. %, and proceeding to sterilization and inoculation;

2d. producing Pleurotus eryngii by culture medium from step 2c;

Step 3: producing cattle feed from the spent Pleurotus eryngii culture medium from step 2, which comprises the following sub-steps:

3a. after harvesting Pleurotus eryngii twice, inoculating the spent Pleurotus eryngii culture medium with feed microbial agents, fermenting the granules for 7 days, and drying the fermented matter to 15 wt. % water content, then pulverizing and sifting the matter through a sieve with mesh size of 40;

3b. producing cattle feeds from the treated Pleurotus eryngii culture medium from step 3a;

Step 4: feeding cattle with mixed feeds supplemented by 30 wt. % of the feed from step 3;

Step 5: producing organic fertilizer from the cattle manure of step 4, which comprises the following sub-steps:

5a. mixing 65% of the cattle manure of step 4 with 30% energy grass in dry weight, composting and retting the mixture, then maintaining the water content to 65 wt. %;

5b. inoculating organic fertilizer starter culture into the mixture from 5a;

5c. fermenting the mixture for 15 days, drying the matter to a moisture content of 15 wt. %, pulverizing and sifting the matter through a sieve with mesh size of 30, spraying nutrient fortified composition, granulating by disk granulation or extrusion granulation method, and packaging.

What is claimed is:

1. An integrated method of cyclic utilization of energy grasses featuring cyclic and integrated use of energy grasses, biogas, edible mushrooms, livestock or poultry feeds, and organic fertilizers, said method comprising the steps of:
(1) generating biogas and biogas residue from said energy grasses;
(2) growing edible mushrooms from said biogas residue;
(3) producing livestock or poultry feeds from said spent mushroom culture medium;
(4) feeding livestock or poultry with said feeds;
(5) producing organic fertilizers from the manure of said livestock or poultry.

2. The method of claim 1, wherein said energy grass is selected from one or more of the following: Neyraudia reynaudiana (Kunth) Keng, Saccharum arundinaceum, Phragmites australis (Cay.) Trin. ex Steud, Miscanthus floridulus (Labill.) Warb. ex Schum. et Laut., Pennisetum purpureum Schumach, Pennisetum sinese Roxb, Pennisetum purpureum cv. Jujuncao, Pennisetum alopecuroides (L.) Spreng, Spartina anglica Hubb., Sorghumpropinqaum (Kunth.) Hitch., Sorghum bicolor L. Moench, silaged Zea mays L, Cymbopogon citratus, Spartina alterniflora Loisel.

3. The method of claim 1, wherein said edible mushroom is one of the following: Pleurotus eryngii, Flammulina velutipes, Volvariella volvacea, Pleurotus ostreatus, Agaricus bisporus, Auricularia auricula, Lentinus edodes, Pleurotus geesteranus, Auricularia polytricha, Tremella fucitormis, Ganoderma lucidum, Hypsizygus marmoreus, Grifola frondosa, and Agrocybe cylindracea.

4. The method of claim 1, wherein said livestock or poultry is one of the following: cattle, sheep, chicken, duck, goose, rabbits, and pigs.

5. The method of claim 1, wherein steps (1)-(5) are accomplished in the following more specific manners:
(1) generating biogas and biogas residue accomplished by first cutting, smashing and ensiling said energy grasses, and then fermenting the energy grasses to generate biogas and biogas residue;
(2) growing edible mushrooms accomplished by first drying said biogas residue, preparing mushroom culture medium from the dry biogas residue, and then growing edible mushrooms using said culture medium;
(3) producing livestock or poultry feeds by first fermenting, drying, smashing, sifting the spent mushroom culture medium, and then producing livestock or poultry feeds from said treated mushroom culture medium;
(4) feeding livestock or poultry with accomplished by administering to the livestock or poultry with said mushroom residue livestock or poultry feeds alone or mixed with other feeds;
(5) producing organic fertilizers accomplished by first mixing said livestock or poultry manure with other organic matters, composting, retting, and inoculating organic fertilizer leavening agent into the mixture, and finally fermenting, drying, smashing, sifting, and spraying nutrient fortified composition to obtain organic fertilizers.
